(19) United States
(12) Patent Application Publication
(10) Pub. No.: US 2014/0212404 A1
(43) Pub. Date: Jul. 31, 2014

(54) Compositions and Methods for Treating Injuries to the Visual System of a Human

(71) Applicant: Khizer Khaderi, Davis, CA (US)

(72) Inventor: Khizer Khaderi, Davis, CA (US)

(21) Appl. No.: 14/080,409

(22) Filed: Nov. 14, 2013

Related U.S. Application Data
(60) Provisional application No. 61/726,216, filed on Nov. 14, 2012.

(51) Int. Cl. A61K 38/51; A61K 9/00

(52) U.S. Cl. CPC A61K 38/51; A61K 9/0048; A61K 9/0051
USPC 424/94.5

(57) ABSTRACT

Methods and compositions for treating injuries to the neural system of a human eye and corresponding structures in the brain are designed to degrade gylcosaminoglycans (GAGs) within chondroitin sulphate proteoglycans (CSPG) using chondroitinase ABC (cABC), preserving neural architecture and protecting the visual system from progressive damage after vascular insult of the optic nerve and/or other portions of the visual system. The methods include delivering the cABC composition by topically applying the composition onto the patient's cornea, stereotactic injections into the related anatomy, delivery by micro-osmotic pumps, delivery by nanoparticles and/or nanocapsules, and delivery through intravitreal implants.

### CROSS-REFERENCE TO RELATED APPLICATIONS

The present specification claims priority to U.S. Provisional Patent Application No. 61/726,216, entitled “Compositions and Methods for Treating Injuries to the Visual System of a Human” and filed on Nov. 14, 2012, which is herein incorporated by reference in its entirety.

### FIELD

The present specification discloses methods of, and compositions for, treating injuries to the neural system of a human eye and corresponding structures in the brain and treating conditions that adversely affect a human's visual system. More particularly, the present specification discloses treating injuries to the neural system of a human eye and corresponding structures in the brain by degrading inhibitory chondroitin sulphate proteoglycans (CSPGs) using compositions comprising chondroitinase ABC.

### BACKGROUND

When a person's visual system is injured, it impairs his or her ability to process visual details. As shown in FIG. 1, and as it pertains to the present specification, the visual system comprises the optic nerve 101, including intraocular (inside the eye) 101a, intraorbital (outside the eye) 101b, and intracanalicular (inside the optic nerve canal) 101c portions, the optic chiasm 103, the optic tract 105, lateral geniculate nucleus 107 (the primary relay center located inside the thalamus of a brain for visual information received from the retina of an eye), optic radiations 109 (a collection of axons from relay neurons in the lateral geniculate nucleus of the thalamus carrying visual information to the visual cortex and which refer to structures as they leave the lateral geniculate nucleus until they reach the occipital cortex), and optical centers in the occipital cortex 111.

Injuries to any portion of the visual system can lead to the formation of a glial scar due to a reactive cellular process which quickly seals off the injured site from healthy tissue, thereby preventing uncontrolled tissue damage, for example, by bacterial invasion. The formation of the glial scar prevents the damage from spreading to other areas of the brain and also enables the affected brain area to heal.

Glial scar formation has both beneficial and detrimental effects, however. Cells within the glial scar secrete certain neuro-developmental inhibitor molecules that prevent complete physical and functional recovery of the affected brain area. The scar poses as a physical barrier to growing neurons, as the scar contains chondroitin sulphate proteoglycans (CSPGs), which are potent inhibitors to the growth and sprouting of neurons. The formation of a glial scar also interferes with endogenous neural repair, such as, formation of new connections, which has been associated with impairments in the repair of the blood brain barrier.

Such injuries may occur to a physical insult or via other physiological processes, including stroke, multiple sclerosis, Alzheimer's disease, Parkinson's disease and other neurodegenerative diseases. For example, a stroke usually occurs when blood flow to a part of the brain is interrupted. Such interruption might occur due to breakage of an artery or blood vessel (hemorrhagic stroke), and may cause the affected part of the brain to be damaged. In most cases, brain damage results in loss of one or more abilities, such as vision, speech, movement, or memory, controlled by the damaged brain part. The extent of disability caused depends upon the extent of damage and the portion of the brain damaged.

A stroke caused by blockage of an artery carrying oxygenated blood from the heart to the brain is termed an ischemic stroke. The blockage of such an artery disrupts the supply of oxygen and other nutrients carried by blood to the brain and also the removal of carbon dioxide and cellular waste. Hence, the brain cells either die or begin to malfunction, thereby causing loss of one or more abilities controlled by the affected brain cells. Ischemic stroke is by far the most common kind of stroke, and may affect people of all ages, including children. Many people with ischemic strokes are older, and the risk of stroke increases with age. The incidence of stroke is expected to rise from approximately 700,000 per year to more than 1,000,000 per year as the population ages. Damage to an area of the brain leads to the formation of a glial scar due to a reactive cellular process, as described above.

Prior art disclosures of the therapeutic use of chondroitinase ABC (cABC) have been made. U.S. patent application Ser. No. 10/698,190 discloses “methods and compositions for inhibiting glial scar formation, methods and compositions for decreasing [glycosaminoglycan] GAG content, methods and compositions for decreasing proteoglycan gene expression, and methods and compositions for promoting neuronal regeneration”.

U.S. patent application Ser. No. 10/877,066 discloses “methods and compositions for rendering a cellular environment permissive to axon regeneration and neural cell transplantation. Methods for stimulating axon regeneration in adult subjects are also disclosed. The methods may comprise contacting a tissue with an agent that prevents glial scar formation, such as by inhibiting reactive astroglial cells, and optionally an agent that increases [B-cell lymphoma 2] bc1-2 protein levels in neural cells”.

U.S. patent application Ser. No. 10/368,809 discloses “a method of inducing neuronal production in a subject, a method of recruiting neurons to a subject's brain, and a method of treating a neurodegenerative condition by administering a neurotrophic factor and an inhibitor of pro-gliogenic bone morphogenetic proteins. Also disclosed is a method of suppressing astrocyte generation and inducing neuronal production in a subject, a method of treating a neurologic condition, and a method of suppressing glial scar formation in a subject by administering an inhibitor of pro-gliogenic bone morphogenetic proteins”.

U.S. patent application Ser. No. 13/058,931 discloses “the use of members of the human hyaluronidase family (endo-beta-acetyl-hexosaminidase enzymes, E.C. 3.2.1.35) for the degradation of chondroitin sulfate (proteoglycans) in the glial scar to promote axonal regrowth in human CNS or spinal cord injury. The [specification] also relates to methods for determining endoglycosidase activity, and in particular of the hyaluronidase/chondroitinase type, in a sample”.

However, the prior art does not provide an effective method of treating injuries to the visual system, including providing anti-inflammatory, neuroprotective and neuroplasticity benefits to the visual system, preserving neural architecture, and generally protecting the visual system from progressive damage after an insult to any portion of the visual system.

Hence, there is a need for methods of, and compositions for, treating injuries to the visual system, including methods and compositions that provide the aforementioned anti-inflammatory, neuroprotective and neuroplasticity benefits to the visual system, preserve neural architecture, and generally protect the visual system from progressive damage after an insult to any portion of the visual system.

### SUMMARY

The present specification discloses methods of, and compositions for, degrading gylcosaminoglycans (GAGs) within the chondroitin sulfate proteoglycans (CSPGs), preserving neural architecture, and protecting the visual system from progressive damage after vascular insult of the optic nerve and/or other portions of the visual system.

In one embodiment, the present specification discloses a method of treating a patient suffering from a condition adversely affecting the patient's visual system by administering to the patient a composition comprising chondroitinase ABC (cABC) wherein the administration of the composition to the patient has at least one of an anti-inflammatory, neuroprotective and/or neuroplasticity therapeutic effect. In various embodiments, the condition is at least one of optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia.

In one embodiment, the composition is administered by topically applying the composition onto the patient's cornea. In various embodiments, the composition is in the form of an aqueous topical solution, ointment, gel, gel-in-situ, drug saturated contact lenses, drug saturated corneal shields, and/or ocular inserts.

In various embodiments, the composition is administered by injection in a form of direct injections into the patient's subconjunctiva, tenon's capsule, sclera, vitreous, cerebral ventricles, intrathecal space, sclera, or optic neurovascular bundle. In one embodiment, the composition is administered by stereotactic injections into the patient's visual system. In one embodiment, the composition is administered by a micro-osmotic pump into the sclera, vitreous, cerebral ventricle, intrathecal space, or directly into the optic neurovascular bundle. In one embodiment, the composition is administered in a form of a nanoparticle and/or nanocapsule injected into the patient's blood, cerebral ventricles, intrathecal space, sclera, vitreous, directly into optic neurovascular bundle, or stereotactically into the patient's visual system. In one embodiment, the composition is administered via intravitreal implants.

In one embodiment, the composition comprises cABC in an amount of 1-100,000 units. In one embodiment, the composition comprises cABC concentrated in a range from 50 to 500 units/mL in a buffering solution.

The present specification further discloses a method of treating a patient suffering from a condition adversely affecting the patient's visual system comprising administering to the patient a composition comprising chondroitinase ABC (cABC) wherein the administration of the composition to the patient has at least one of an anti-inflammatory, neuroprotective and/or neuroplasticity therapeutic effect and wherein the composition comprises cABC in an amount of 1-100,000 units and wherein the composition comprises cABC concentrated in a range from 50 to 500 units/mL in a buffering solution.

In various embodiments, the condition is at least one of optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia.

In one embodiment, the composition is administered using hydrogels.

The present specification further discloses a composition effective to treat a patient suffering from a condition adversely affecting the patient's visual system, wherein said composition comprises chondroitinase ABC (cABC) in an amount of 1-100,000 units concentrated in a range from 50 to 500 units/mL in a buffering solution and wherein the administration of the composition to the patient has at least one of an anti-inflammatory, neuroprotective and/or neuroplasticity therapeutic effect on at least one of the patient's cornea, anterior chamber, iris, pupil, ciliary body, lens, vitreous humor, retina, choroids, optic nerve, optic chiasm, optic tract, lateral geniculate nuclei, optic radiations, visual cortex, or visual association cortex.

In various embodiments, the condition is at least one of optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia.

In one embodiment, the composition is administered using a gel-foam impregnated with cABC.

In various embodiments, the composition is prepared to be administered by injection in a form of direct injections into the patient's subconjunctiva, tenon's capsule, sclera, vitreous, cerebral ventricles, intrathecal space, sclera, or optic neurovascular bundle or is prepared to be administered by stereotactic injections into the patient's visual system. In one embodiment, the composition is prepared to be administered by a micro-osmotic pump into the sclera, vitreous, cerebral ventricle, intrathecal space, or directly into the optic neurovascular bundle. In one embodiment, the composition is prepared to be administered in a form of a nanoparticle and/or nanocapsule injected into the patient's blood, cerebral ventricles, intrathecal space, sclera, vitreous, directly into optic neurovascular bundle, or stereotactically into the patient's visual system. In one embodiment, the composition is prepared to be administered via intravitreal implants.

The aforementioned and other embodiments of the present specification shall be described in greater depth in the drawings and detailed description provided below.

### BRIEF DESCRIPTION OF THE DRAWINGS

These and other features and advantages of the inventions disclosed herein will be further appreciated, as they become better understood by reference to the detailed description when considered in connection with the accompanying drawings:

FIG. 1 is an illustration of a visual system of a human;

FIG. 2A is an image, using Wisteria floribunda agglutinin (WFA) staining, of a brain affected by a stroke being treated with cABC, in accordance with one embodiment of the present specification;

FIG. 2B is an image, using Wisteria floribunda agglutinin (WFA) staining, of a brain affected by a stroke being treated with cABC and a hydrogel, in accordance with one embodiment of the present specification;

FIG. 2C is an image, using chondroitin-4-sulfate (C4S) staining, of a brain affected by a stroke being treated with cABC, in accordance with one embodiment of the present specification; and,

FIG. 2D is an image, using chondroitin-4-sulfate (C4S) staining, of a brain affected by a stroke being treated with cABC and a hydrogel, in accordance with one embodiment of the present specification.

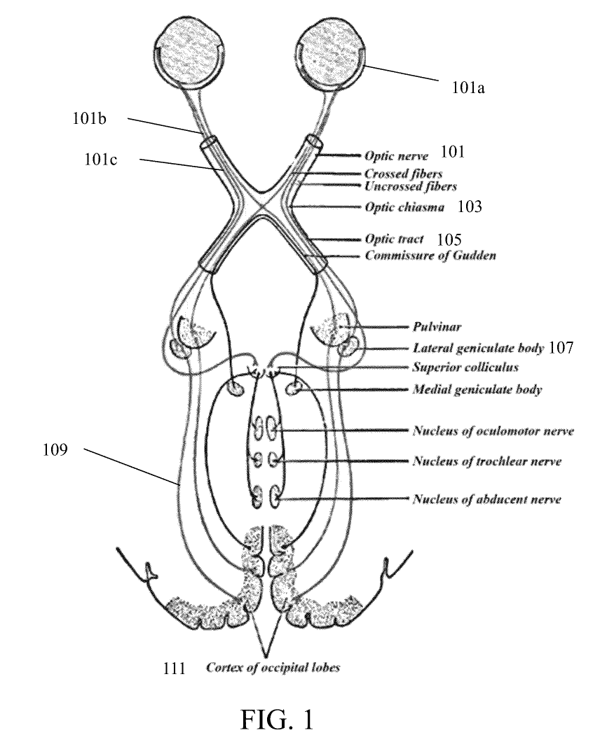

### DETAILED DESCRIPTION

The present specification discloses methods of, and compositions for, introducing the enzyme chondroitinase ABC (cABC) into a portion of the visual system for effectively degrading inhibitory chondroitin sulphate proteoglycans (CSPGs).

The present specification discloses multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the presently disclosed inventions are to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

The term “patient” shall refer to a human mammal who has incurred a physical or vascular insult to its visual system, due, for example, to an accident, assault, tumor, bone abnormality, or surgery, and/or whose visual system is subject to degradation due to an on-going neuro-degenerative condition, such as stroke, multiple sclerosis, Alzheimer's disease or Parkinson's disease.

The term “anti-inflammatory” shall refer to a physiological effect in which, relative to a pre-treatment state, a patient's biological response to harmful stimuli such as damaged cells, also referred to as inflammation, is decreased due to the treatments disclosed herein. Anti-inflammatory effects shall be particularly measured by reference to the amount and extent of optic nerve inflammation, which may be caused by trauma, raised intracranial pressure, a disruption of cerebral spinal fluid, autoimmune diseases, demyelinative inflammation, optic neuritis, and/or traumatic optic neuropathies. The degree of inflammation may be quantified by detecting the presence of, and measuring an amount of, inflammatory markers present in serum or cerebrospinal fluid.

The term “neuroprotective” shall refer to a physiological effect in which, relative to a pre-treatment state, the adverse effects of a chronic disease are prevented or slowed by halting or slowing the loss of optic nerve axons, optic ganglion cells, or other portions of the visual system. Such chronic diseases include, but are not limited to, Parkinson's disease, Alzheimer's disease, Huntington's disease, stroke, glaucoma, anterior ischemic optic neuropathy, and/or cancer.

The term “neuroplasticity” shall refer to a physiological effect in which, relative to a pre-treatment state, a patient's visual system is capable of modifying or otherwise changing in order to account for injuries while still maintaining, to the best extent possible, visual system functionality. Enabling or improving neuroplasticity is of particular importance when dealing with conditions in which neural networks along the optic nerve pathway are disrupted or mal-developed, such as with amblyopia.

In one embodiment, a patient suffering from optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia is treated with a cABC composition in order to achieve at least one of an anti-inflammatory, neuroprotective and/or neuroplasticity therapeutic effect.

In one embodiment, the composition comprises cABC in an amount of 1-100,000 units depending on the degree of enzymatic need. One unit is defined as the quantity of enzyme that catalyses the formation of 1 μmole of unsaturated disaccharide from chondroitin sulfate C per minute at 37° C. and a pH equal to about 8.0. Because cABC is hydrophilic, it can be concentrated in a range from 50 to 500 units/mL. The composition further comprises a buffering solution, including, but not limited to, bicarbonate-carbonate buffers (pH 9.2-10.8), phosphate buffers (pH 5.8-8.0), or citrate buffers (pH 3.0-6.2). cABC is highly specific for degradation of the inhibitory GAG side-chains of CSPGs.

In one embodiment, the specific concentration of cABC in the composition and administration methodology is dependent upon where, along the optic nerve pathway, the composition is delivered and for what condition. For example, when attempting to deliver anti-inflammatory, neuroprotective or neuroplasticity therapeutic benefits to portions of a patient's visual system which are in confined spaces, such as the intraocular optic nerve, the concentration of cABC may be higher relative to situations where the portion of a patient's visual system in need of anti-inflammatory, neuroprotective or neuroplasticity therapeutic benefits is not in a confined space.

In another embodiment, the cABC composition is administered to a patient suffering from optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia in order to achieve at least one of an anti-inflammatory, neuroprotective and/or neuroplasticity therapeutic effect by topically applying the composition onto the patient's cornea. The composition is in the form of an aqueous topical solution, an ophthalmic drop solution, ointment, gel, gel-in-situ, drug saturated contact lenses, drug saturated corneal shields, and/or ocular inserts. It should be appreciated that an ophthalmic drop solution may require higher concentrations and/or a pairing with a penetration agent when compared to intra-ocular or intra-cranial injections. In one embodiment, the composition has a concentration of cABC of 1-100,000 units/microliter at a dose of 10-1,000 units per injection.

In another embodiment, the cABC composition is administered to a patient suffering from optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia in order to achieve at least one of an anti-inflammatory, neuroprotective and/or neuroplasticity therapeutic effect by injection. Injections may take the form of direct injections into the patient's subconjunctiva, tenon's capsule, sclera, vitreous, cerebral ventricles, intrathecal space, sclera, or optic neurovascular bundle. Injections may also be made stereotactically into the point of interest along the visual system.

In particular, the composition may be administered by a micro-osmotic pump into the sclera, vitreous, cerebral ventricle, intrathecal space, or directly into the optic neurovascular bundle. Alternatively, the composition may be administered in the form of a nanoparticle and/or nanocapsule injected into the patient's blood, cerebral ventricles, intrathecal space, sclera, vitreous, directly into the optic neurovascular bundle, or stereotactically into a point of interest in the visual system. Alternatively, the composition may be administered via intravitreal implants.

In another embodiment, the cABC composition is administered to a patient suffering from optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia in order to achieve at least one of an anti-inflammatory, neuroprotective and/or neuroplasticity therapeutic effect using hydrogels. A hydrogel is defined by a system in which two part synthetic liquid systems are mixed resulting in the cross-linking of materials to form a soft, flexible, and lubricious hydrogel. The hydrogel linkages gradually hydrolyze over time. The composition may be a hydrogel with cABC incorporated therein and delivered into the vitreous, cerebral ventricle, directly into or around the optic neurovascular bundle, or stereotactically into a point of interest in the visual system.

In another embodiment, the cABC composition is administered to a patient suffering from optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia in order to achieve at least one of an anti-inflammatory, neuroprotective and/or neuroplasticity therapeutic effect using a gel-foam impregnated with cABC and delivered to the conjunctival pouch, optic nerve, or directly into a portion of interest within the visual system. In one embodiment, cABC coated polymers are surgically placed onto the optic nerve or a point of interest within the visual system.

In another embodiment, the present specification provides for the synergistic use of neuronal progenitor cells (NPC) with cABC delivered via hydrogel directly into a stroke cavity via a patient's ocular system. cABC possesses anti-inflammatory, neuroprotective, and potential neuroplasticity effects which may be used to treat stroke, in addition to the indications described above.

In an exemplary application, a hydrogel may be synthesized from acrylate-modified hyaluronic acid, laminin protein, and heparin sulfate. This synthesized hydrogel is in a liquid form to which cABC and/or NPC are added. Next, a cross-linker (polyethylene glycol dithiol) is added to the hydrogel and cABC and/or NPC mixture and the resultant mixture is infused via a stereotactic surgery into the stoke cavity. Stereotactic surgery is a minimally invasive form of surgical intervention which makes use of a three-dimensional coordinate system to locate small targets inside the body and to perform on them some action such as ablation, biopsy, lesion, injection, etc. The injected mixture forms a solid structure within the stroke cavity in approximately half an hour. Lyophilized cABC is diluted in normal saline to a concentration of 0.2 units/μL and a dosage comprising 0.1 units/mouse stroke of cABC is injected along with hydrogel into a stroke cavity.

In an experimental set up, an ischemic stroke in the left striatum of a mouse was generated via left carotid artery ligation followed by stereotactic infusion of a potent vasoconstrictor, which is an agent that causes a narrowing of an opening of a blood vessel. Next, human NPCs were infused into the stroke cavity of the mouse. One hundred thousand human intermediate progenitor NPCs (IP-NPCs) were transplanted per mouse, and each mouse received daily immunosuppression against the T-lymphocytes with cyclosporin via subcutaneous mini-osmotic pumps at a dose of 25 μL/hr. A minimal survival rate of NPC was observed leading to the conclusion that for successful xeno-transplantation (the process of transplanting organs or tissues between members of different species) in mice, the natural killer cells must be eliminated, as well as the T-lymphocytes. It was also observed that modifying the inhibitory properties of the glial scar alters tissue reorganization after stroke, and elimination of the inhibitory CSPGs allows a more robust endogenous repair mechanism and therefore a return to near normal central nervous system (CNS) architecture after stroke.

Two weeks after selected intervention, each mouse was perfused, post fixed, and cryoprotected (protected from damage caused due to freezing). Coronal sections, 40 μm thick were cut through the striatum with the cryostat (an apparatus for taking very fine slices of tissue while it is kept very cold) and were stained for: Wisteria floribunda agglutinin (WFA) (Sigma 1:10); chondroitin-4-sulfate (C4S) (Millipore 1:1000); glial fibrillary acidic protein (GFAP) (Sigma 1:100); and, ionized calcium-binding adapter molecule 1 (IBA-1) (Wako 1:1000). It was observed that striatal stroke causes progressive tissue atrophy (wasting away due to degeneration of cells) of affected striatum and secondary dilatation of the ipsilateral ventricle (belonging on the same side of the body as the affected striatum). Next, the size of the striatum and the ventricles were quantified by the circumferential area of the affected side. These were then compared with the contralateral side. Images were obtained on a confocal microscope. Immunohistochemical (IHC) stains were analyzed as pixel levels of fluorescence above background and divided by the overall area of the ipsilateral section. These were then averaged and compared with background staining from a contralateral section.

WFA binds to chondroitin sulfate (CS) of the glial scar allowing visualization of net-like structures of perineuronal nets (PNNs). A positive WFA staining represents intact glial scar whereas a negative WFA staining represents cABC-mediated digestion of CSPGs. In rats, digestion of PNNs by using cABC reactivates a visual critical period. In an exemplary set up, it was observed that digestion of PNNs in the visual cortex well after the closure of a critical period, such as postnatal day 70, reactivated critical period plasticity and allowed ocular dominance shift to occur in rats. However, the effects of monocular deprivation in the reactivated case are not as strong as monocular deprivation during a normal critical period. Additionally, in adult rats that had been monocularly deprivated since youth, digestion of PNNs brought about a full structural and functional recovery (recovery of ocular dominance, visual acuity, and dendritic spine density). However, this recovery only occurred once the open eye was sutured to allow the cortical representation of the deprived eye to recover.

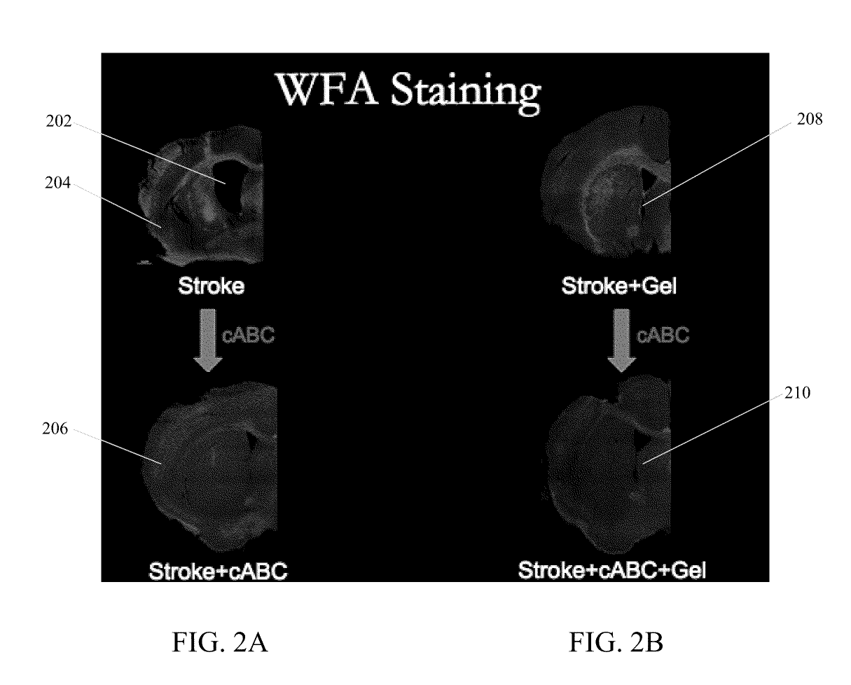

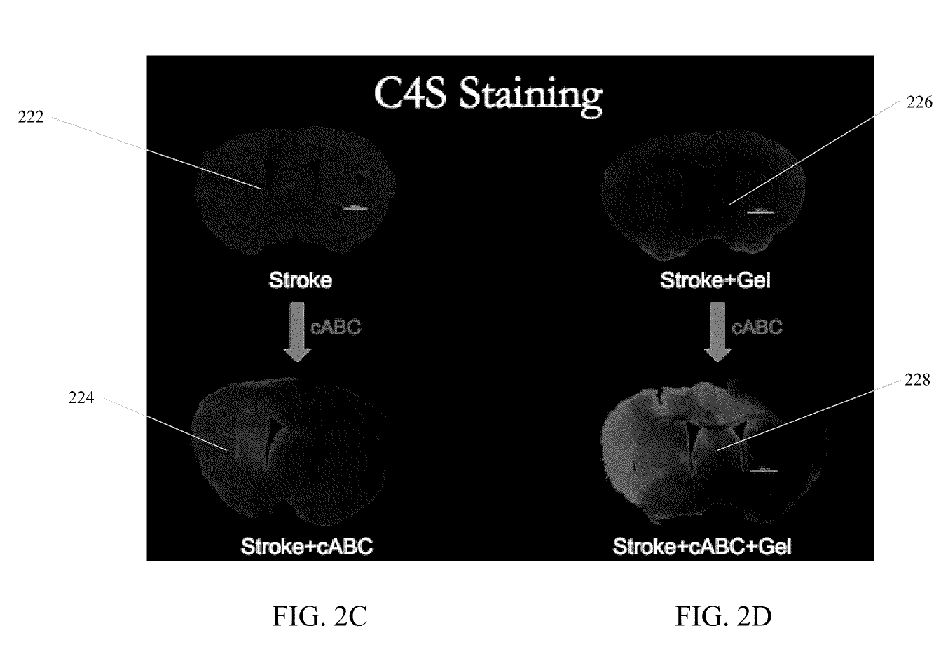

FIG. 2A depicts a brain 202 affected by a stroke being treated with cABC, in accordance with an embodiment of the present specification. Referring to FIG. 2A, a brain 204 comprising a stroke cavity 202, upon being treated with cABC, appears as brain 206 on being stained with WFA. FIG. 2B depicts a brain affected by a stroke being treated with cABC and a hydrogel, in accordance with an embodiment of the present specification. As illustrated, brain 208 affected by a stroke and injected with a hydrogel appears as brain 210 upon being treated with cABC and stained with WF.

In an embodiment, chondroitin-4-sulfate (C4S) adheres to the exposed protein core of CSPG that is only available if CS-GAG side chains have been cleaved by cABC. FIG. 2C depicts a brain 222 affected by a stroke being treated with cABC, in accordance with an embodiment of the present specification. As illustrated, a brain 222 affected by a stroke upon being treated with cABC appears as brain 224 on being stained with C4S. FIG. 2D depicts a brain 226 affected by a stroke being treated with cABC and a hydrogel, in accordance with an embodiment of the present specification. As illustrated, brain 226 affected by a stroke and injected with a hydrogel appears as brain 228 upon being treated with cABC and stained with C4S.

When comparing percentage sizes of a contralateral striatum relative to treatment approaches, it is expected that it would be greatest when using cABC and hydrogel, followed by use of cABC only, and followed by use of hydrogel only. When comparing percentage sizes of a ventriculam relative to different treatment approaches, it is expected that it would be greatest when using only hydrogel, followed by using only cABC, and followed by using cABC and hydrogel.

In an embodiment of the present specification, chondroitinase ABC (cABC) is concentrated to 0.2 units/μL, dissolved and maintains enzymatic activity in a hydrogel, and is injected in a dose of 0.1 units in a stroke cavity of a brain, thereby leading to effective degradation of the inhibitory CSPGs within the post-stroke glial scar formed in the stroke cavity. Further, the present invention provides that treating a stroke with cABC maintains normal brain architecture by preserving striatal size and preventing post-stroke ventriculomegally. Yet further, in accordance with the present specification, treating a brain stroke with cABC leads to a significant increase in reactive astrocytic response while decreasing inflammatory response.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of treating a patient suffering from a condition adversely affecting the patient's visual system comprising administering to the patient a composition comprising chondroitinase ABC (cABC), wherein said administration of said composition to said patient has at least one of an anti-inflammatory, neuroprotective and/or neuroplasticity therapeutic effect on the visual system.

2. The method of claim 1 wherein the condition comprises at least one of optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia.

3. The method of claim 1 wherein the composition is administered by topically applying the composition onto the patient's cornea.

4. The method of claim 3 wherein the composition is in the form of an aqueous topical solution, ointment, gel, gel-in-situ, drug saturated contact lenses, drug saturated corneal shields, and/or ocular inserts.

5. The method of claim 1 wherein the composition is administered by injection in a form of direct injections into the patient's subconjunctiva, tenon's capsule, sclera, vitreous, cerebral ventricles, intrathecal space, sclera, or optic neurovascular bundle.

6. The method of claim 1 wherein the composition is administered by stereotactic injections into the patient's visual system.

7. The method of claim 1 wherein the composition is administered by a micro-osmotic pump into the sclera, vitreous, cerebral ventricle, intrathecal space, or directly into the optic neurovascular bundle.

8. The method of claim 1 wherein the composition is administered in a form of a nanoparticle and/or nanocapsule injected into the patient's blood, cerebral ventricles, intrathecal space, sclera, vitreous, directly into optic neurovascular bundle, or stereotactically into the patient's visual system.

9. The method of claim 1 wherein the composition is administered via intravitreal implants.

10. The method of claim 1 wherein the composition comprises cABC in an amount of 1-100,000 units concentrated in a range from 50 to 500 units/mL in a buffering solution.

11. A method of treating a patient suffering from a condition adversely affecting the patient's visual system comprising administering to the patient a composition comprising chondroitinase ABC (cABC) wherein the administration of the composition to the patient has at least one of an anti-inflammatory, neuroprotective and/or neuroplasticity therapeutic effect on at least one of the patient's cornea, anterior chamber, iris, pupil, ciliary body, lens, vitreous humor, retina, choroids, optic nerve, optic chiasm, optic tract, lateral geniculate nuclei, optic radiations, visual cortex, or visual association cortex and wherein the composition comprises cABC in an amount of 1-100,000 units and wherein the composition comprises cABC concentrated in a range from 50 to 500 units/mL in a buffering solution.

12. The method of claim 11 wherein the condition comprises at least one of optic nerve inflammation caused by trauma, raised intracranial pressure, a disruption of cerebral spinal fluid, autoimmune diseases, deymyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia.

13. The method of claim 11 wherein the composition is administered using hydrogels.

14. A composition effective to treat a patient suffering from a condition adversely affecting the patient's visual system, wherein said composition comprises chondroitinase ABC (cABC) in an amount of 1-100,000 units concentrated in a range from 50 to 500 units/mL in a buffering solution and wherein the administration of the composition to the patient has at least one of an anti-inflammatory, neuroprotective and/or neuroplasticity therapeutic effect.

15. The composition of claim 14 wherein the condition comprises at least one of optic nerve inflammation caused by trauma, raised intracranial pressure, a disruption of cerebral spinal fluid, autoimmune diseases, demyelinative inflammation, optic neuritis, and/or traumatic optic neuropathies, Parkinson's disease, Alzheimer's disease, Huntington's disease, stroke, glaucoma, anterior ischemic optic neuropathy, cancer, and/or amblyopia.

16. The composition of claim 14 wherein the composition is administered using a gel-foam impregnated with cABC.

17. The composition of claim 14 wherein said composition is prepared to be administered by injection in a form of direct injections into the patient's subconjunctiva, tenon's capsule, sclera, vitreous, cerebral ventricles, intrathecal space, sclera, or optic neurovascular bundle or is prepared to be administered by stereotactic injections into the patient's visual system.

18. The composition of claim 14 wherein said composition is prepared to be administered by a micro-osmotic pump into the sclera, vitreous, cerebral ventricle, intrathecal space, or directly into the optic neurovascular bundle.

19. The composition of claim 14 wherein said composition is prepared to be in a form of a nanoparticle and/or nanocapsule injected into the patient's blood, cerebral ventricles, intrathecal space, sclera, vitreous, directly into optic neurovascular bundle, or stereotactically into the patient's visual system.

20. The composition of claim 14 wherein said composition is prepared to be administered via intravitreal implants.
